(19) United States
(12) Patent Application Publication
(10) Pub. No.: US 2021/0329990 A1
(43) Pub. Date: Oct. 28, 2021

(54) ARTICLE FOR PERSONAL PROTECTIVE EQUIPMENT USING AN ELECTROCEUTICAL SYSTEM

(71) Applicant: Ion Defense Technologies, LLC, Hamilton, MT (US)

(72) Inventors: Mark Holler, Hamilton, MT (US); David Morrow, Waco, TX (US)

(21) Appl. No.: 17/239,355

(22) Filed: Apr. 23, 2021

Related U.S. Application Data
(60) Provisional application No. 63/015,893, filed on Apr. 27, 2020.
(60) Provisional application No. 63/048,559, filed on Jul. 6, 2020.

(51) Int. Cl. A41D 13/11; A61L 9/22

(52) U.S. Cl. CPC A41D 13/113; A61L 9/22; A41D 13/1192

(57) ABSTRACT

An electroceutical system integrated into an article of personal protective equipment (PPE) like a face mask. The system consists of an electrochemical cell consisting of an anodic zone capable of serving as an anode, a cathodic zone capable of serving as a cathode and, optionally, a selectively conductive switch zone electrically couplable to and separating the anodic and cathodic zones and serving to switch on and off current flow between the anodic and cathodic zones.

### BACKGROUND

The inventive subject invention relates generally to personal protective equipment (“PPE”) that serves to filter air from an exterior side of the article to a body facing side of the article. For example, the inventive subject matter may be used as a protective face mask or respirator, a buff, a bandage or wound covering, and similar uses. More particularly, the inventive subject matter is directed to an “electroceutical system” integrated into an article of personal protective equipment (PPE) like a face mask. The system uses at least one electrochemical cell consisting of at least an anodic zone capable of serving as an anode, a cathodic zone capable of serving as a cathode and optionally selectively conductive switch zone electrically couplable to and separating the anodic and cathodic zones and serving to selectively switch on and off current flow between the anodic and cathodic zones. A face mask or respirator will be used hereinafter as a representative article of personal protective equipment.

Facial masks have been used for hundreds of years to protect medical workers and people in close contact with others when there is danger of infection.

Masks are crucially needed in hospitals, residential care facilities, workplaces, sporting events, concerts, large shopping centers, airplanes and public transportation. They are needed by people who are healthy to diminish potential infection from breathing the same air or receiving spray (i.e., sneeze). They also protect those around a person who is infected but asymptomatic.

In 2020, Hong Kong, a city of 7.5 million people, instituted mandatory wearing of masks and social distancing very early in the COVID-19 pandemic. The city infection rate was very low compared to places that did not take early precautions. There is a world-wide need for masks for the general public.

A traditional mask (e.g. 3M Particulate Respirator 8210) seals effectively around the nose and mouth and is comfortable enough to wear for extended periods. A traditional mask works by filtering particles and droplets. For example, a N95 mask filters 95% of fine particles. Traditional masks are made from cotton and are considered one-use. Traditional masks do not kill or neutralize Infectious Agents (IA), nor do they wick moisture and heat away from the user. Unfortunately, they are the most commonly available. Worldwide there is a limited production capacity for one-use traditional masks. Advantageously, articles according to the inventive subject matter may be configured so that they are washable and reusable many times, and they can be mass produced using existing technology for rapid supply expansion. Testing for such masks may be conducted as described in ASTM F2100-11, which is incorporated herein by reference.

Various designs and configurations for face masks have been previously proposed. One class of masks uses a filter network to trap the pathogens. These face masks include the surgical type masks commonly worn in hospitals. One example is described in U.S. Pat. No. 7,044,993 to Bolduc entitled “Microbicidal air filter.” Bolduc discloses a system that employs an immobilization network of fibers having antimicrobial agents incorporated and molecularly bonded into its structure. Another class of masks include those that employ filter canisters to trap the pathogens. One example is described in U.S. Pat. No. 6,681,765 to Wen entitled “Antiviral and antibacterial respirator mask.” Wen discloses a system that employs a filtration apparatus containing both an active stage and passive stage filter in the mask.

Many metals are known to have oligodynamic action (biocidal action by a metallic substance). For instance, silver has been a known antibiotic agent for at least 6,000 years. It was used to store food and prevent spoiling in ancient Babylon, as evidenced by archaeological finds. Silver has been used in wound dressings for many decades and was particularly important before the discovery of penicillin. Silver fabrics are used by the military to prevent fungus and bacterial infection on the battlefield in such products as bandages, socks and underwear. Copper is a well-known oligodynamic element, either in raw form or combined with other metals (e.g., brass). Research has shown that copper or brass elements (e.g., bed frames) in hospital environments substantially reduce transmission of IA. The mechanism for this reduction occurs when bacteria and viruses are destroyed or inactivated by encountering ions of copper or copper compounds.

In recent time, textiles impregnated or treated with very fine silver bits or other oligodynamic particles been developed. In some cases, the particles are on the nanoscale.

Numerous metals and metallic compounds may emit ions that disrupt bacteria via three pathways: 1. Respiration, 2. Replication, and 3. Cell wall synthesis. Likewise, metals or metallic compounds may disrupt viruses by disassociating the fatty membrane surrounding the RNA. The metallic ions also disrupt the proteins that may surround a virus which allow it to attach to and penetrate a living cell. Metals such as copper or silver are much less likely to promote the development of resistant IA than traditional antibiotics that typically target only one of these pathways. Antimicrobial silver has been used extensively in hospitals for decades with no clinically relevant cases of antibiotic resistance.

The H1N1 virus is thought to have caused the 1918 influenza pandemic and swine flu outbreak in 2009. Silver nanoparticles have been shown to inhibit viruses such as H1N1, as cited here: “Our data suggest that silver nanoparticles exert anti-HIV activity at an early stage of viral replication, most likely as a viricidal agent or as an inhibitor of viral entry. Silver nanoparticles bind to gp120 in a manner that prevents CD4-dependent virion binding, fusion, and infectivity, acting as an effective viricidal agent against cell-free virus (laboratory strains, clinical isolates, T and M tropic strains, and resistant strains) and cell-associated virus. Besides, silver nanoparticles inhibit post-entry stages of the HIV-1 life cycle.” Mode of antiviral action of silver nanoparticles against HIV-1 in the Journal of Nanobiotechnology, 2010 Jan. 20. Humberto H Lara, Nilda V Ayala-Nuñez, Liliana Ixtepan-Turrent, and Cristina Rodriguez-Padilla.

Unfortunately, existing PPE filtration articles have various deficiencies that need to be overcome. While some may be effective at entrapping IA, the pathogen may remain in the filter in a dangerous active state. This causes risk to the PPE user handling and using the article. Typically, therefore, the article needs to be discarded after a single or limited use. During use of the articles, particularly masks and respirators, moisture and heat can build-up on the body-facing side. This causes users discomfort. Further, it may cause the articles to degrade faster, which also limits their use to single or limited use. For these and other reasons, there is a substantial need for improved PPE articles.

### SUMMARY

The inventive subject matter overcomes the deficiencies in the prior art by providing an electroceutical system integrated into an article of personal protective equipment (PPE) like, for example, a face mask. The system uses an electrochemical cell consisting of an anodic zone capable of serving as an anode, a cathodic zone capable of serving as a cathode and a selectively conductive switch zone electrically couplable to and separating the anodic and cathodic zones and serving to selectively switch on and off current flow between the anodic and cathodic zones. The energized electrochemical system imparts electrical properties to the zones, i.e., the zones are electrically energized structures. Infectious agents in the zones are subject to inactivation or disruption from the electrical effects. When the electrochemical cell is based on a textile wherein the yarns or fibers of the textile have material properties that define the anodic and/or cathodic regions, the textile may be referred to as an “electroceutical fabric”. All forms of textile construction are contemplated for use herein, including, wovens, knits, and non-wovens, as webs, matts, and felts of fiber.

In other aspects, the inventive subject matter may incorporate oligodynamic materials into a layer of material that is included in a PPE article. In yet another aspect, the inventive subject matter may include an exterior surface that repels environmental droplets and moisture laden with IA. In a further aspect, the PPE article may include a moisture management feature. In another aspect, the PPE article may include a thermal regulation feature for conductively dissipating heat generated by the user or otherwise present on the user side of the article. In still another aspect, the inventive subject matter may combine one or more of the foregoing features with a feature that kills or disrupts IA via an energizable layer or layers that is energized by a mechanism other than foregoing electrochemical cell. Any one or more layers may be energized via direct electrical current, electrostatic charge, charged particles, resistive heating, or IR heating.

The article may be constructed of common or available materials so that it can be reused 25 times or more.

In a possible embodiment, the inventive subject matter is directed to an article comprising a filtration mask. The mask is made from one or more layers. It has an external side (or major surface) for facing side the environment, and an internal side (or major surface) for facing the body of a user. The mask is air permeable over aa facial area covering at least the mouth and/or nose, the permeable facial area includes an energizable structure in the nature of an electrochemical cell consisting of an anodic fabric zone capable of serving as an anode, a cathodic fabric zone capable of serving as a cathode, and a selectively conductive switch zone electrically couplable to and separating the anodic and cathodic zones and serving to selectively switch on and off current flow between the anodic and cathodic zones. The switch may be moisture activated. In certain embodiments, the zones are all fabric or textile materials. The anodic zone and cathodic zone may be on opposite, lateral sides of the facial area, and the switch zone may be disposed between and adjacent to the anodic and cathodic zones. The switch zone may be moisture activated to initiate and sustain current flow between the anodic and cathodic zones. The switch zone may be an electrically insulating fabric that can absorb moisture, e.g., moisture from the intended user's breath or from skin perspiration.

In some embodiments, the switch may be activated by contact with a user's skin that is relatively dry, the skin providing the electrical bridge between the anodic and cathodic zones, and the switch zone in the mask being a passive insulator that serves to prevent conductivity between the anodic and cathodic zones when the mask is not worn.

The foregoing and other embodiments are described in more detail in the following detailed descriptions and the figures.

The following is a description of various inventive lines under the inventive subject matter. The appended claims, as originally filed in this document, or as subsequently amended, are hereby incorporated into this Summary section as if written directly in.

The foregoing is not intended to be an exhaustive list of embodiments and features of the inventive subject matter. Persons skilled in the art can appreciate other embodiments and features from the following detailed description in conjunction with the drawings.

### BRIEF DESCRIPTION OF THE DRAWINGS

The appended figures show embodiments according to the inventive subject matter, unless noted as showing prior art.

FIGS. 1A-16 schematically show a 2-layer filtration article with a first layer having an internal side against a user's skin and an adjacent outer layer having an external side to open air.

FIGS. 2A-2B schematically show a filtration article implemented as a face mask or respirator, with the FIG. 2A showing the article in place on a user's face, the view including an oval cutaway to illustrate the three layers, and with FIG. 2B showing a cross section of the three layers in more detail.

FIG. 3 schematically shows a filtration article embodying an electrochemical cell system.

### DETAILED DESCRIPTION

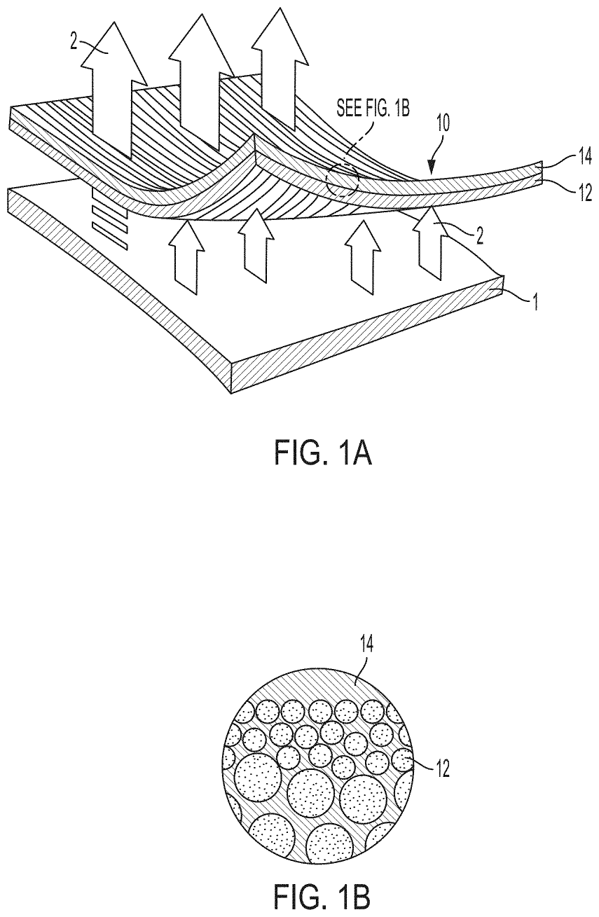

Representative embodiments according to the inventive subject matter are shown in FIGS. 1A-3, wherein the same or generally similar features share common reference numerals.

In general, the articles of PPE contemplated herein include a first, bodyside or inner layer 12, that is oriented against a user's skin. A second layer 14 is adjacent the first layer or spaced apart from it by one or intermediate layers 16 and is oriented towards the external environment. One or both of the first and second layers generally would have a filtration function for capturing external particles in a porous network. The porosity can be defined to physically exclude particles, including IA, from passing through to the surface of the body side layer towards the user's skin or inhalation passages, or other body parts needing protection. Filtration media include woven, knit, and non-woven textiles. They may also include non-textiles like polymer foams. The filtration media may be any combination of the foregoing constructs. Filter media is well known in the art and commercially available. For masks and respirators, the porosity and density of the filtration media are controlled to allow for sufficient airflow for respiration while still entrapping particles. Masks and other filtration articles may include mechanical one-way valves to help vent gases 2 and vapor 2 from the bodyside of the article while allowing environmental air to enter.

The inventive subject matter is generally directed to an energizable structure for disrupting or inactivating IA in PPE articles. It is particularly suited for use in face masks and respirators that include area that covers at least the mouth and/or nose of the intended user.

In a possible embodiment, the inventive subject matter is directed to an article comprising a filtration mask. The mask is made from one or more layers. It has an external side for facing the environment, and an internal side for facing the body of a user. The mask is air permeable over at least a facial area over the mouth and/or nose, the permeable facial area includes an energizable structure in the nature of an electrochemical cell consisting of an anodic fabric zone capable of serving as an anode, a cathodic fabric zone capable of serving as a cathode, and optionally a selectively conductive switch zone electrically couplable to and separating the anodic and cathodic zones and serving to switch on and off current flow between the anodic and cathodic zones.

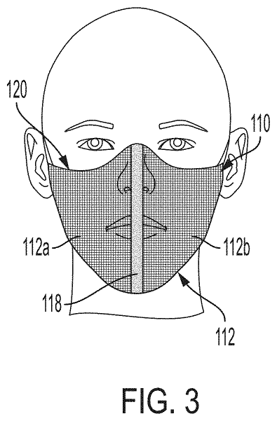

FIG. 3 illustrates one possible, non-limiting example of a face mask with anodic and cathodic zones. In this embodiment, the zones are all fabric or textile materials. The zones can be arranged in different ways. In certain embodiments, current flows between the zones when the mask becomes moistened by contact with water or other electrolytic solvent. Means of moistening the zones include vapors from a user's exhalation, perspiration, environmental moisture (humidity), or user-controlled, manual wetting of the zones.

In the example shown, the mask is bifurcated into anodic and cathodic zones. The mask may include a conductive perimeter zone or band that is in direct or indirect electrical contact with left and right zones to conductively couple the left and right zones. The perimeter zone may fully or partially encircle the mask. It may be made of an anodic or a cathodic fabric like one of the left or right anodic or cathodic zones and participate in the redox reaction. Or it may be made of a different conductive fabric or other conductive structure. The conductive perimeter zone can be part of the fabric construction, e.g., a hem, border, or rand.

As illustrated, there optionally is a central switch zone that runs from the top edge of the mask to the bottom edge. In this example, the switch zone is a non-conductive fabric in a dry state. When wetted, it becomes conductive. It is disposed between and adjacent to left and right anodic and cathodic zones. It does not matter on which side one such zone is; whatever a given side is, the opposite side will have the opposite polarity.

The anodic zone and cathodic zone may be on opposite, lateral sides of the facial area, and the switch zone may be disposed between and adjacent to the anodic and cathodic zones. The switch zone may be moisture activated to initiate and sustain current and/or ion flow between the anodic and cathodic zones via a redox (reduction-oxidation) reaction between dissimilar materials on either side of the switch zone. The switch zone may be an electrically insulating fabric that can absorb moisture, e.g., moisture from the intended user's breath or from skin perspiration. In general, the dissimilar materials may have different electronegativities, e.g., metals and salts thereof where the base metals on the opposite sides of anodic and cathodic zones have different electronegativities. Advantageously, many of the oligodynamic molecules and materials disclosed and contemplated herein for disrupting IA may also serve as the base molecules or materials for the anodic and cathodic zones.
As one non-limiting example, in the mask of FIG. 3, the right lateral side of the mask could comprise a fabric base material, e.g., 95% by weight polyester fabric with a 5% by weight grid or web of anodic or cathodic fibers, yarns or other conductive material or structure that is interwoven, interknit, coated, printed or otherwise overlaid on or embedded in the base fabric. The anodic material could be, for example, copper, silver, or carbon fiber. The left lateral side of the mask could have a similar construction but with a different base anodic/cathodic material than used on the opposite right side. One example of a suitable structure would be incorporation of the base anodic and cathodic materials in or on yarns that are interwoven or interknit with the base fabric in a grid or web construction like used in ripstop fabrics. In addition to polyester, the base fabric for any zone may be nylon, rayon, or other well-known natural or synthetic fabrics.

Some specific, non-limiting, examples of fabrics believed suitable for use as anodic or cathodic zones include:

Carbon Fiber Based Fabrics
Triange 90% Poly 8% lycra 2% carbon fiber
Miti (https://www.mitispa.com/) with smart silver treatment 90% poly 7% lycra 3% carbon fiber
Miti rayon 96% Rayon 4% CF. (This CF configuration is a known loop or circuit weave, because it crosses over in a vertical and horizontal pattern.)
Poly carbon fiber

Silver Based Fabrics
Borgini silver T2 97% poly 3% Silver salt imbedded yarn (https://www.borgini.it/)
Silver Mesh 90% poly 8% lycra 2% silver yarn
Nylon silver

The non-conductive zone can be the same base material as the lateral sides but without any anodic or cathodic material as is used on the lateral side zones. This way a single base material can be used for all zones to simplify construction. For example, a panel of fabric containing the anodic material may contain the same base material as a panel of fabric containing the cathodic material. In some embodiments, a border of the base material extends entirely or partially around a perimeter of each panel. A segment of the border of one such panel can be joined (e.g., sewn, glued, etc.) with a corresponding segment of the border of the other panel, defining a non-conductive zone positioned between opposing regions of cathodic material and anodic material. The non-conductive zone so positioned can define a switch zone, as described herein.

The switch zone may be moisture activated to initiate a redox reaction between the adjacent anodic and cathodic zones. In some embodiments, the switch may be activated by contact with a user's skin that is relatively dry, the skin providing the electrical bridge between the anodic and cathodic zones, and the switch zone in the mask being a passive insulator that serves to prevent conductivity between the anodic and cathodic zones when the mask is not worn.

The electrochemical cell(s) used in the mask or other article may activate by any number of electrolytic solvents, as noted elsewhere, to facilitate redox reactions in the fabric causing ion and/or electron movement in fabric zones that are capable of disrupting IA. The electrochemical cells may be configured as galvanic, voltaic electrolytic, or fuel cells, as are known in the art. Additional elements to support any such cells may include electrical conductors and salt bridges that electrically or chemically couple different zones.

Advantageously, as seen in the embodiment of FIG. 3, only a single electrochemical cell consisting of the lateral halves 112a, 112b of the mask is needed to cover both airways. Also, the single cell may extend to and along the perimeter of the mask or other PPE article, allowing for energization not just at airways but also at perimeter edges where air could leak in. The single electrochemical cell also makes construction simplified.

In some embodiments, a single electrochemical cell extends over at least 25%, or 50%, or 75% or 90% of the surface area of the mask or other article.

While FIG. 3 shows the anodic and cathodic zones on lateral halves 112a, 112b of the mask to substantially cover airways and mask perimetrical edges for energization of the entire covered area, other arrangements are also possible. For example, the zones could be arranged on top and bottom halves of the mask. Multiple electrochemical cells could also be arranged on the mask. IA will likely concentrate on facial areas corresponding to air passages or air exchange areas, one or more electrochemical cells should be arranged where there is substantial air exchange like areas corresponding to mouth and/or nose areas of the intended users face.

The inventive subject matter contemplates other means of switching the mask on and off or controlling its state of energization. For example, the mask's electrochemical cells could be stored with a peel-off non-conductive layer that prevents activation, e.g., moisture activation, when the mask is stored. Another possibility is an ampule-like structure that holds an electrolytic solvent that can be broken by the user to selectively release the solvent and activate the mask. Yet another possibility is a bridge area between anodic and cathodic zones that could be a receiver for a removable conductive element, e.g., a strip of conductive metal that when in place closes the circuit between the anodic and cathodic zones, and when removed, opens the circuit. The received could be a pocket or a surface connector like a hook and loop type surface connector.

Similarly, the mask could be rechargeable by making the anodic and cathodic zones modular. For example, the mask could include at each zone a receiver for a replaceable pad of anodic or cathodic material to restore the masks ability to energize at the zones. Similarly, the mask could be configured with an onboard energy source, e.g., a battery, or to couple with an external energy source to reverse the redox reaction that occurred in the electrochemical cell and restore it to a charged state.

In certain embodiments, the inventive subject matter advantageously combines certain functional features that provide a multimodal system for preventing IA from passing through the filtration article, helping to inactivate the IA, and providing comfort to the user by moisture management and thermal regulation. The articles are formed from an innovative selection and arrangement of structures that synergistically operate to more effectively protect users and provide the user's comfort. Advantageously, a single structure may provide multiple functions. Or, by a scheme of selectively arranging layers, different advantageous effects can be achieved. Certain of the following structures may be selected to achieve desired effects and objectives:
(1) a structure with oligodynamic materials for inactivating the selected IA;
(2) a structure (e.g., a woven, knit, non-woven textile structure) that includes carbon fibers or particles (which may be referred to as a “carbonized structure”) for moisture management and/or conductive, convective or radiative cooling;
(3) a knit or woven structure having yarns in a denier gradient that causes wicking of water from the internal side of the mask toward the external side;
(4) a carbonized structure that is energized, or energizable, that subjects entrapped IA to current, charged particles, electrostatic discharge, resistive heating and/or disruptive electromagnetic energy (this structure may be the same as or different from the carbonized structure for moisture/thermal management);
(5) a hydrophobic structure forming an external surface of the external side for repelling droplets and other moisture from absorbing into the surface;
(6) An electroceutical system integrated into a PPE article like a mask, consisting of an electrochemical cell consisting of at least one anodic zone capable of serving as an anode, at least one cathodic zone capable of serving as a cathode. The mask may optionally include a selectively conductive switch zone electrically couplable to and separating the anodic and cathodic zones and serving to switch on and off current flow between the anodic and cathodic zones.

The filtration articles according to the inventive subject matter may include some or all the foregoing six primary functional features, in any permutations.

In many applications, the structures are woven, knit, or non-woven textile structures. However, other non-textiles may also serve as structures. For example, polymer foams or sheet materials may support functional features contemplated herein. For example, a porous polymer sheet could provide air permeability and have oligodynamic materials associated with it. An open cell foam could similarly serve as a substrate for functional features. By controlling cell size, the foam could also serve as a filtration medium. Polymers and foams can also provide a structural backbone for shaping an article of PPE to a better conform to a body part. Or they can serve as bonding layers interconnecting other layers.

The filtration article also may be constructed so that it may be reused and washed or laundered. And it can be cost effectively produced.

In one possible embodiment, the inventive subject matter is directed to a protective face mask that cost-effectively offers a broad spectrum of antimicrobial protection using fabrics treated with one or more oligodynamic metals and/or salt of an oligodynamic metal. The oligodynamic material may be in the form of a thread, a small particle, an impregnation of fabric threads, or a film.

A first functional feature serves to kill, destroy, disrupt or otherwise make the IA inactive so it cannot harm an intended user. As one example of forming an oligodynamic structure in the article, metallic elements are impregnated into, coated on, or otherwise associated with a fabric so that they can inactivate viruses and bacteria. Numerous tests show a neutralizing capacity of over 99% for 600 bacteria and virus species using certain oligodynamic materials. Articles according to the inventive subject matter may use commercially available fabrics that are treated with a nanoparticle metal solution or incorporate nanoparticles of metal directly into the yarn or fibers where it is woven or knit at a fabric mill.

The nanoparticles can be incorporated into any structure of the article so that a single structure has multiple functional features. For example, a mill could weave or knit the fabric containing both the nano-metal and carbon fiber for the carbonized structure. In either case there will be millions of metallic oligodynamic nanoparticles incorporated into each device.

A second functional feature helps provide comfort to the user and/or helps prevent degradation of the article by managing moisture and heat that may build up on the internal side of the article. For example, in a mask, heat and moisture build up on the internal side as the user exhales. To deal with this, the article may incorporate carbon fiber in yarns that are woven or knit into a fabric used as a structure in the article. Carbon fiber incorporated into a worn garment provides excellent heat and moisture management and has been used in sports clothing for this reason. The user of the article benefits from a more comfortable product and thus would be more likely to wear a mask that incorporates carbon fiber yarns or strands into the fabric.

A third functional feature is another moisture management tool that operates by wicking moisture from the internal side of the article. The article may be constructed with a wicking system. For example, a denier gradient fabric wicks moisture away from the wearer, enhancing user comfort and article durability. Denier gradient fabrics comprise multiple fabric layers having different deniers that scale in one direction. The denier gradient causes moisture to travel by capillary action from the larger denier fabric side to a smaller denier fabric side. U.S. Pat. No. 4,733,546, issued Mar. 29, 1988, to Toda, titled “KNITTED FABRIC FOR CLOTHING,” incorporated herein by reference, describes one such variable denier gradient fabric (“Toda”). In particular, Toda describes a fabric having a surface layer yarn of a certain denier, such as, for example, 1.0 denier to 2.5 denier. The back layer of the fabric would be preferably 50% or more larger than the surface layer denier. The voids between the larger denier fibers of the back layer would be larger than the voids between the smaller denier fibers in the surface layer. Thus, capillary action would cause moisture to move from the back layer towards the outermost most, external-facing layer, and away from the user's skin. The mask may be designed to drive the moisture away from the skin to an intermediate layer or all the way to the outermost, external-facing layer, where the moisture can evaporate into surrounding air. This action has been found useful in designing moisture management fabrics.

This action is also described in U.S. Pat. No. 6,381,994B1, filed Jun. 28, 2001 by Young-Kyu Lee titled “Method for making fabric with excellent water transition ability”. Refer to FIG. 1 for a drawing of this fabric technology. Patent Number: EP0766520 to Laycock and Walker (expired) describes a technique to create a denier gradient fabric, “A multilayer breathable cloth of a clothing garment, said cloth comprising at least two separate layers interlocked, the layers having differing deniers so as to provide a denier gradient through the thickness of the fabric, at least one of the layers being of a woven structure, wherein the finer denier layer is located at the outside of the garment.” A denier gradient is illustrated in FIGS. 1A-1B, which show layer 12 subdivided into a first layer 12 of a larger gradient and second layer of a smaller gradient. FIGS. 1A-16 also show the movement of water 2 or moisture 2 away from a skin 1 adjacent the first layer to the exterior surface of the second layer.

A fourth functional feature provides an energized or energizable structure that helps to inactivate IA. A carbonized structure can provide a substrate for such function. Carbon fiber is generally anisotropic and conducts electricity in a linear fashion along the length of the carbon fiber as opposed to transversely across the width of the fiber. However, carbon fiber has a potential for a small amount of transverse electrical current flow. Treating fabric (which contains carbon fiber strands) with a solution containing metal nanoparticles will bring the two dissimilar materials into contact. Depending on the anodic index of the metal used there will certainly be some current or ion movement, essentially creating a low-power battery or electrostatic effects. It is believed that this electrical potential may substantially enhance the oligodynamic effectiveness of the mask because ion exchange is known to disrupt bacteria and viruses.

Other possibilities based on the conductivity of carbon include, having carbonized structures that serve as an anode and cathode. The material properties of the carbonized structures may be varied to provide different electrical or material properties. For example, one carbonized structure could incorporate oligodynamic particles on a surface to provide desired electrical effects, as well as oligodynamic inactivation of IA. The structures can be connected to a power source like a battery which when switched causes current to flow. Filtration medium entrapping IA may be disposed between the anode and cathode layers so that the current passing between helps inactivate the IA.

In another possibility embodiment, the anode and cathode layers are separated by a dielectric filter medium so that the layer can store an electrical charge. A discharge event will cause a capacitive, electrostatic effect across the dielectric layer intermediate the carbonized layers. If the dielectric layer is also the filter medium, the discharge will help disrupt entrapped IA.

As another possibility, the energized structure could be conductive fabric that is attached to a power source, causing resistive heating in the layer. The layer could be different from or the same as the filtration medium. If different, it only needs to be sufficiently close for heat transfer.

A fifth functional feature is a hydrophobic outer surface that may actively repel mist, liquid droplets, and other moisture. In one of many possible examples, the outer layer is constructed with a manufactured fabric (e.g., polyester) that is impregnated with Durable Water Repellent (DWR) compounds. This DWR stops liquids at the outer surface of the fabric. Hydrophobic DWR, while somewhat durable, can wash out after many launderings but can be renewed with commonly available products (e.g., Nikwax). Another possibility is to make the outer surface from a textile made with hydrophobic yarns or fibers, e.g., expanded PTFE membrane. Such membrane could also be a backing to a more durable outer layer.

Another notable advantage of the inventive articles contemplated herein, which unlike throw-away masks, the article may be constructed of durable materials that can be washed and reused. The fabrics and sewing materials used may be those that are common in many industries, including sports and casual clothing crafting.

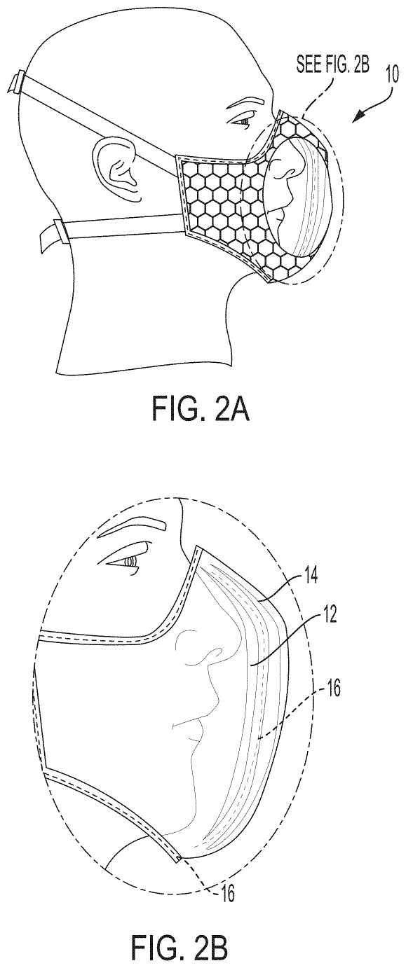

FIG. 1 illustrates a two-layer filtration article wherein each layer may embody one or more of the functional features discussed herein. The article shown is just to illustrate a layering scheme, and the article may be embodied into any kind of PPE. FIGS. 2A-2B shows a filtration article in the form of mask or respirator. In this example, the filtration article includes an optional third layer sandwiched between the first and second layers. The inventive subject matter is not limited to an article of 1, 2 or 3 layers. It may have any greater number of layers that provide the desired functional features disclosed herein.

The layers contemplated herein may be discrete layers that are bonded, fused or otherwise joined together using known techniques. Or two or more layers may be unitary structures that are not formed of discrete structures. For example, knit and woven structures can be formed in multiple, seamlessly joined layers with the layers varying in terms of materials, yarn deniers, and/or crossover picks and/or loop densities. Similarly, non-wovens can have different layers monolithically formed by varying the size, denier, or material laid out or deposited in the formation process. Accordingly, a single physical layer may embody one or more functional features disclosed herein.

In the example embodiments of FIGS. 1A-2B, the two primary layers are an outer first layer and an inner second layer. Depending on use, the device may have one or more intermediate layers. FIGS. 2A-2B show an example intermediate layer (the third layer). For example, an intermediate layer may be a bonding layer, e.g., a thin polyester foam or glue, or a thermally fusible polymer layer. It may be a fabric-backing that adds strength and body. Some fusible polymer materials could serve as a bonding layer and a backing layer.

In the case of a mask or respirator like seen in FIGS. 2A-2B, the article forms a covering over at least the inhalation passages of the user's face, namely the nose and mouth. The covering may sit flush against the face, like a surgical mask. Or, as shown, it may have a three-dimensional shape that forms a void in front of the passages. Whatever form, the mask may have a perimeter that is intended to provide a seal against the skin so that environmental air, and whatever particles the air carries, must pass through the filtration layer(s) of the mask and any other desired functional layer. The perimeter may be an elastic or rubbery material that conform to the user's face and helps seal against it.

The covering may be a standalone article, or it may part of a larger article. For example, it could be part of a full head covering, a jacket hood, and balaclava, a neck gaiter, etc. It could be a permanent part of any such article, or it could be removable, replaceably attached using any known system of joining, e.g., hook and loop fasteners, snaps, buttons, zippers and slide seals, glue, etc. When removably attached, the article to which it attaches can be configured with an opening design that fits against the mask so that there are no gaps around the mask, thereby providing a more effective environmental seal.

In the example of FIGS. 2A-2B, the article is finished with a sewn binding material. The binding may contain an oligodynamic material so that there is better protection at all possible points of entry around the entire perimeter of the article.

Referring to FIGS. 2A-2B, the layers will be discussed in more detail. The discussion is intended to be non-limiting and only to illustrate one of many possible embodiments at a more detailed level.
Layer 1:
This outside layer 14 in this example may have some or all the following components:
A) Textile base that may have carbon fiber, silver, copper or other oligodynamic material incorporated (e.g., woven, knit, fused, printed, deposited, melted, admixed) into the textile or the constituent yarns or fibers. The base or another component may also serve as a filtration medium to physically entrap particles. The fabric material and construction may be any one of those discussed elsewhere herein.
B) Durable water repellent finish on the exterior surface.
C) Surface treatment (e.g., spray, wet dip, etc.) on the exterior and/or interior surface or otherwise incorporating silver nanoparticles or similar oligodynamic material.
Layer 2:
This inside layer 12 in this example may have some or all the following components:
A) Textile base with carbon fiber.
B) Denier gradient moisture control textile. The fabric material and construction may be any one of those discussed elsewhere herein.
C) Textile or fibers thereof treated with or otherwise incorporating silver nanoparticles or other oligodynamic material, the same or different from the oligodynamic material on the first layer. The fabric material and construction may be any one of those discussed elsewhere herein.
Layer 3 (optional):
A) Polyester foam that may be treated with antimicrobial agents
B) Glue
C) Fusible material and/or backing material (e.g., a non-woven polymer material to bond adjacent layers or to give form to the mask)
D) Filtration medium to physically entrap particles.

Fabric materials to construct this invention can include but are not limited to: single product or blends of rayon, polyester, spandex, cotton, wool, elastane, polyamide, carbon fiber yarn, silk, cashmere, silver, copper, nylon, bamboo, hemp, and blends of any one or more of the foregoing materials. These fabrics may be used in any one or more layers of a filtration article, discussed in more detail below. Carbon fibers are often woven into athletic clothing for the wearer's comfort, and for this device, is appropriate for a face mask.

A carbon fiber is a long, thin strand of material. It may have a range of diameters. In some cases, expected to be suitable for use in the inventive subject, it has a diameter of about 0.005-0.010 millimeter. However, the inventive subject is not necessarily limited to that range and smaller or larger diameters may be useful, depending on the selected application. While not intending to be bound to any theories or principles, it is understood that the carbon atoms in carbon fiber are bonded together in microscopic crystals that are more or less aligned parallel to the long axis of the fiber. The crystal alignment makes the fiber incredibly strong for its size. Several thousand carbon fibers may be twisted together to form a yarn, which may be used by itself or woven into a fabric. The resulting fabric can be a drapable product suitable for garments and filtration articles. The carbon fibers may also be blended with other known yarn materials. As used herein, a carbon fiber yarn or textile or fabric refers to any such construction that has at least 2% carbon fiber or particles by volume. Carbon fiber yarns may be woven or knit into one of the fabric types discussed below.

Fabric types to construct this invention may include knit, woven, and/or non-woven textiles. The textiles may include but are not limited to: single knit, double knit, plaited, jersey, lame, mesh, tricot, fuse, ripstop, felting, laminating, bonding, canvas, pile, Jacquard, dobby, gauze, raschel tabby, twill, satin, buckram, cambric, casement, cheese cloth, chiffon, chintz, corduroy, crepe, denim, drill, flannel, gabardine, georgette, khadi, lawn, mulmul, muslin, poplin, sheeting, taffeta, tissue, velvet, mousseline, organdie/organza, leno, aertex, madras muslin, and aida.

The inventive subject matter may use a Durable Water Repellent (DWR) finish on outer layers to repel IA contained in droplets expelled (e.g., by coughing or sneezing) into the environment surrounding a mask or other PPE article. Thus, the inventive subject matter provides a first layer of protection pre-filtration layer of protection that helps keep IA from entering the mask. DWRs are non-polar or hydrophobic compositions. They are well-known and widely available in the general textile industry. They have been applied to a variety of textiles to inhibit water absorption.

Because water is a polar molecule in the liquid phase it tends to clump into droplets on the hydrophobic DWR finish. These droplets are relatively easy to stop on a fabric face. In the vapor phase, water molecules are smaller and more energized therefore they can move easily through many textiles, both woven and non-woven, even those having a DWR finish. Thereby, articles according to the inventive subject may be finished with a DWR technology to repel droplets and other moisture while providing some venting (breathability) of moisture from the inward facing side of the article. An advantage of a DWR finish on an oligodynamic filtration article is it extends the articles usefulness and efficacy by eliminating moisture which can degrade the physical structure and features of the article.

Durable Water Repellent (DWR) finishes may include but are not limited to single products, polymers or blends using wax(s), oils, fluorocarbons, fluoropolymers, silicon, non-wax hydrocarbons, perfluorobutanesulfonic acid, perfluorooctanoic acid and related compounds. DWR application methods may include but are not limited to dipping, spraying, chemical vapor deposition and related techniques or combination of methods.

A variety of oligodynamic materials may be applied to or incorporated into textiles (or constituent fibers or yarns used to make the textiles) to kill, destroy, neutralize, or otherwise disrupt IA like bacteria and viruses. These include, but are not limited to, silver, mercury, copper, iron, lead, zinc, bismuth, gold, aluminum, platinum, palladium, iridium, tin, and antimony. Oligodynamic metal salts can include, but are not limited to, silver acetate, silver carbonate, silver chloride, silver citrate, silver cyanide, silver hydroxide, silver nitrate, silver nitrite, silver oxide, silver phosphate, silver sulfate, mercury acetate, mercury carbonate, mercury chloride, mercury citrate, mercury cyanide, mercury hydroxide, mercury nitrate, mercury nitrite, mercury oxide, mercury phosphate, mercury sulfate, copper acetate, copper carbonate, copper chloride, copper citrate, copper cyanide, copper hydroxide, copper nitrate, copper nitrite, copper oxide, copper phosphate, copper sulfate, iron acetate, iron carbonate, iron chloride, iron citrate, iron cyanide, iron hydroxide, iron nitrate, iron nitrite, iron oxide, iron phosphate, iron sulfate, lead acetate, lead carbonate, lead chloride, lead citrate, lead cyanide, lead hydroxide, lead nitrate, lead nitrite, lead oxide, lead phosphate, lead sulfate, zinc acetate, zinc carbonate, zinc chloride, zinc citrate, zinc cyanide, zinc hydroxide, zinc nitrate, zinc nitrite, zinc oxide, zinc phosphate, zinc sulfate, bismuth acetate, bismuth carbonate, bismuth chloride, bismuth citrate, bismuth cyanide, bismuth hydroxide, bismuth nitrate, bismuth nitrite, bismuth oxide, bismuth phosphate, bismuth sulfate, gold acetate, gold carbonate, gold chloride, gold citrate, gold cyanide, gold hydroxide, gold nitrate, gold nitrite, gold oxide, gold phosphate, gold sulfate, aluminum acetate, aluminum carbonate, aluminum chloride, aluminum citrate, aluminum cyanide, aluminum hydroxide, aluminum nitrate, aluminum nitrite, aluminum oxide, aluminum phosphate, aluminum sulfate, platinum acetate, platinum carbonate, platinum chloride, platinum citrate, platinum cyanide, platinum hydroxide, platinum nitrate, platinum nitrite, platinum oxide, platinum phosphate, platinum sulfate, palladium acetate, palladium carbonate, palladium chloride, palladium citrate, palladium cyanide, palladium hydroxide, palladium nitrate, palladium nitrite, palladium oxide, palladium phosphate, palladium sulfate, iridium acetate, iridium carbonate, iridium chloride, iridium citrate, iridium cyanide, iridium hydroxide, iridium nitrate, iridium nitrite, iridium oxide, iridium phosphate, iridium sulfate, tin acetate, tin carbonate, tin chloride, tin citrate, tin cyanide, tin hydroxide, tin nitrate, tin nitrite, tin oxide, tin phosphate, tin sulfate, antimony acetate, antimony carbonate, antimony chloride, antimony citrate, antimony cyanide, antimony hydroxide, antimony nitrate, antimony nitrite, antimony oxide, antimony phosphate, antimony sulfate, or combinations thereof. Suitable oligodynamic metals or oligodynamic metal salts could be readily obtained or prepared by persons of skill in the art and incorporated into a filtration article as described herein.

Contrary to conventional thinking, in some applications, depending on the oligodynamic material, some moisture in a layer of a mask or other article with oligodynamic material may be desirable. For example, the moisture may help activate an oligodynamic metal into a more active form or help disperse it through a layer or layers of the article for wider distribution and more effective action. To provide both user comfort and such moisture activation, the internal portion of the mask may use a moisture management structure like a denier differential or carbonized fabric to move moisture away from the face or other body part to a more outward portion where oligodynamic material is to be moisture activated. For example, the moisture management feature may be present at an internal surface and the oligodynamic feature may be at an intermediate layer or portion.

U.S. Pat. No. 8,183,167 to Delattre et al is incorporated here by reference. The Abstract reads: “Substrates that exhibit antimicrobial and/or antifungal characteristics that persist through the useful life of the substrate, and more particularly textile substrates infused with or covalently bound to well-dispersed antimicrobial nanoparticles, such as silver and/or copper nanoparticles, which exhibit persistent and demonstrable bactericidal, bacteriostatic, fungicidal, fungistatic behavior through numerous wash cycles. Methods of manufacturing such substrates are also provided.”

The oligodynamic material may be in the nature of nanoparticles. A nanoparticle is usually defined as a particle whose diameter is between 1 and 100 nanometers. Nanoparticles are usually distinguished from “fine particles”, sized between 100 and 2500 nanometers, and “coarse particles”, ranging from 2500 to 10,000 nanometers. They are a subclass of the colloidal particles, which are usually understood to range from 1 to 1000 nanometers. The properties of nanoparticles often differ markedly from those of larger particles of the same substance. Since the typical diameter of an atom is between 0.15 and 0.6 nm, a large fraction of the nanoparticle's material lies within a few atomic diameters from its surface. Therefore, the properties of that surface layer may dominate over those of the bulk material. This effect is particularly strong for nanoparticles dispersed in a medium of different composition, since the interactions between the two materials at their interface also becomes significant. Ref: Batista, Carlos A. Silvera; Larson, Ronald G.; Kotov, Nicholas A. (9 Oct. 2015). “Nonadditivity of nanoparticle interactions”. Science. 350 (6257): 1242477. doi:10.1126/science.1242477. ISSN 0036-8075. PMID 26450215.

A benefit of nanoparticles is that millions or more of them can be applied to and impregnated into a square meter of fabric. For example, silver nanoparticles have a very high surface area, thus the chance of contact with an IA is very high. The IA may contact fixed silver-based oligodynamic materials fixed to the textile substrate or the IA may encounter metallic ions that are generated when the metal is exposed to moisture. In a mask, this moisture would come from the person exhaling through it.

As discussed above, a filtration article according to the inventive subject matter may be composed of one or more layers of specialized fabrics that provide multiple functions alone or collectively.

When worn on the face, the user breathes in and out through the fabric layer or layers including oligodynamic material, IA in the inhaled air will contact the oligodynamic materials that are on or in the fabric. Laboratory tests have shown that over 600 species of bacteria and viruses can be neutralized or destroyed by encountering metallic elements (e.g., copper, silver). When various kinds of IA are trapped in an article according to the inventive subject matter, it is believed that disinfection/decontamination rates of over 99% can be achieved using ISO 18184 antiviral and ISO 20743 antibacterial standards. Disinfection usually should start within a few minutes of moisture from exhalation or other source electrolytically activating the cathodic and anodic zones in the mask or other article.

Persons skilled in the art will recognize that many modifications and variations are possible in the details, materials, and arrangements of the parts and actions which have been described and illustrated in order to explain the nature of the inventive subject matter, and that such modifications and variations do not depart from the spirit and scope of the teachings and claims contained therein.

All patent and non-patent literature cited herein is hereby incorporated by references in its entirety for all purposes.

As used herein, “and/or” means “and” or “or”, as well as “and” and “or.” Moreover, any and all patent and non-patent literature cited herein is hereby incorporated by references in its entirety for all purposes.

The principles described above in connection with any particular example can be combined with the principles described in connection with any one or more of the other examples. Accordingly, this detailed description shall not be construed in a limiting sense, and following a review of this disclosure, those of ordinary skill in the art will appreciate the wide variety of systems that can be devised using the various concepts described herein. Moreover, those of ordinary skill in the art will appreciate that the exemplary embodiments disclosed herein can be adapted to various configurations without departing from the disclosed principles.

The previous description of the disclosed embodiments is provided to enable any person skilled in the art to make or use the disclosed innovations. Various modifications to those embodiments will be readily apparent to those skilled in the art, and the generic principles defined herein may be applied to other embodiments without departing from the spirit or scope of this disclosure. Thus, the claimed inventions are not intended to be limited to the embodiments shown herein, but are to be accorded the full scope consistent with the language of the claims, wherein reference to an element in the singular, such as by use of the article “a” or “an” is not intended to mean “one and only one” unless specifically so stated, but rather “one or more”.

All structural and functional equivalents to the elements of the various embodiments described throughout the disclosure that are known or later come to be known to those of ordinary skill in the art are intended to be encompassed by the features described and claimed herein. Moreover, nothing disclosed herein is intended to be dedicated to the public regardless of whether such disclosure is explicitly recited in the claims. No claim element is to be construed as “a means plus function” claim under US patent law unless the element is expressly recited using the phrase “means for” or “step for”.

The inventors reserve the right to claim, without limitation, at least the following subject matter.

1. A PPE article, comprising:
one or more layers, the article having an external side for facing side the environment, and an internal side for facing the body of a user, and wherein the article includes an energizable structure comprising an electrochemical cell consisting of an anodic zone capable of serving as an anode, and a cathodic zone capable of serving as a cathode.

2. The article of claim 1, wherein the article comprises a face mask that is air permeable over at least a facial area corresponding to the mouth and/or nose of an intended user.

3. An article comprising a filtration mask, the mask comprising:
one or more layers, the article having an external side for facing side the environment, and an internal side for facing the body of a user, and wherein the article air permeable over at least a facial area over the mouth and/or nose, the permeable facial area including an energizable structure comprising an electrochemical cell consisting of an anodic fabric zone capable of serving as an anode, a cathodic fabric zone capable of serving as a cathode, and a selectively conductive switch zone electrically couplable to and separating the anodic and cathodic zones and serving to switch on and off current flow between the anodic and cathodic zones.

4. The article of claim 1 wherein the anodic zone and cathodic zone are on opposite, lateral sides of the facial area, and further comprising a switch zone that is disposed between and adjacent to the anodic and cathodic zones, wherein the switch zone is a selectively conductive zone electrically couplable to and separating the anodic and cathodic zones and serving to switch on and off current flow between the anodic and cathodic zones.

5. The article of claim 3 wherein the switch zone is moisture activated to initiate and sustain current flow between the anodic and cathodic zones.

6. The article of claim 3 wherein the switch zone comprises an electrically insulating fabric that can absorb moisture.

7. The article of claim 1 wherein one or both of the anodic zone and cathodic zone comprises an oligodynamic structure.

8. The article of claim 7 wherein the oligodynamic structure comprises a textile structure.

9. The article of claim 8 wherein the anodic and cathodic zones comprise a knit, woven or non-woven structure comprising yarns or fibers.

10. The article of claim 9 wherein the yarns or fibers have coated or embedded oligodynamic particles sufficiently exposed so that they can act as anodic or cathodic and/or oligodynamic elements.

11. The article of claim 10 wherein the particles comprise silver or copper particles or salts or other compositions based thereof.

12. The article of claim 2 further including a first carbon fiber or carbon particle structure (a “carbonized structure”) in one or more of the anodic and cathodic zones.

13. The article of claim 12 wherein the carbonized structure comprises a moisture management and/or conductive cooling structure.

14. The article of claim 12 wherein the carbonized structure comprises an energized or energizable structure.

15. The article of claim 1 wherein the article comprises a mask or respirator sized and shaped to surround and cover at least all the inhalation passages of an intended user.

16. The article of claim 1 wherein the article is made of materials and has a construction sufficiently durable to withstand at least 25 cycles of washing or laundering under conditions typical of home laundering.

17. The article of claim 1 wherein the article has a porosity sufficient to entrap various IA, including corona viruses and other prevalent pathogenic viruses.

18. The article of claim 3 wherein a single electrochemical cell is configured with a surface area to cover at least an area around the air passages of the intended user.

19. The article of claim 1 wherein a single electrochemical cell is disposed along some or all the perimetrical edges of the article.

20. The article of claim 19 wherein a single electrochemical cell extends over at least 25% of the surface area of the article.

21. The article of claim 19 wherein a single electrochemical cell extends over at least 50% of the surface area of the article.

22. The article of claim 19 wherein a single electrochemical cell extends over at least 75% of the surface area of the article.

23. The article of claim 19 wherein a single electrochemical cell extends over at least 90% of the surface area of the article.

24. The article of claim 1 further comprising a conductive perimetrical zone conductively coupling to the anodic and/or cathodic zone in the presence of an electrolytic solvent.

25. The article of claim 1 wherein one or more of the zones have a modular construction for removably replacing the anodic, cathodic, and/or switch zone material.
